SEQUENCE LISTING (1 sequence)

SEQ ID NO: 1 - DNA, 200 bp, synthetic construct
  source 1..200: mol_type = other DNA
  regulatory 1..200: regulatory_class = promoter
agttttcacttgtcccaccaagtgtgtctcttgattttaggctggttttgctactaatttctctaatttgctaaattttagtatgagataaaaatattaattttaatgtattattttggtggtctaatatgtcttactttttcaatgagtgacacatttttaagtactgtatttattctgcaatcaagacacaaactaag

(19) United States
(12) Patent Application Publication
(10) Pub. No.: US 2023/0374590 A1
(43) Pub. Date: Nov. 23, 2023

(54) METHODS AND REAGENT FOR DIAGNOSING BIPOLAR DISORDER

(71) Applicant: Massachusetts Institute of Technology, Cambridge, MA (US)

(72) Inventors: Elly Nedivi, Cambridge, MA (US); Baovi N Vo, Cambridge, MA (US)

(21) Appl. No.: 17/932,132

(22) Filed: Sep. 14, 2022

Related U.S. Application Data
(60) Provisional application No. 63/244,065, filed on Sep. 14, 2021.

(51) Int. Cl. C12Q 1/6883; C12Q 1/6869; C12Q 1/6897

(52) U.S. Cl. CPC C12Q 1/6883; C12Q 1/6869; C12Q 1/6897; C12Q 2600/156

(57) ABSTRACT

A 200 bp section of the CPG2 promoter region is responsive to lithium treatment for bipolar disorder (BD). Compositions and methods for the detection and treatment of BD have been developed which employ sequencing the CPG2 promoter region from the genomic DNA of a subject to identify single nucleotide polymorphisms (SNPs) within the promoter region of the CPG2 gene and then use in vitro transcriptional assays to assess their effects on the expression of the CPG2 protein. Compositions and methods for screening therapeutic agents for treating one or more symptoms of BD in a subject are also provided. The methods may identify subjects having BD, or at risk of BD, and provide effective treatment regimens. In some embodiments, the methods can be used to monitor the success of treatments at the molecular level and provide improved treatment regimens for prophylactic and therapeutic applications.

### CROSS REFERENCE TO RELATED APPLICATIONS

This application claims priority to U.S. Provisional Application No. 63/244,065, filed in the United States Patent and Trademark Office on Sep. 14, 2021, by Massachusetts Institute of Technology, naming inventors Elly Nedivi and Baovi N Vo, incorporated by reference herein.

### REFERENCE TO SEQUENCE LISTING

The Sequence Listing submitted as an xml file named MIT_23591_P_US.xml”, created on Sep. 14, 2022, and having a size of 2,601 bytes, is hereby incorporated by reference pursuant to 37 C.F.R § 1.834(c)(1).

### FIELD OF THE INVENTION

The invention is generally directed to diagnosing and predicting treatment efficacy for bipolar disorder, and in particular, using transcriptional regulation of CPG2 as a molecular indicator of bipolar disorder risk and response to therapeutic agents.

### BACKGROUND OF THE INVENTION

Bipolar disorder (BD) is a common, chronic mood disorder characterized by recurrent episodes of mania and depression. BD interferes with normal function and is associated with high rates of comorbidity, major depression, alcohol and drug abuse, and cardiovascular diseases. The lifetime prevalence is estimated at 1-4% of the population, and the high mortality rate makes BD a major public health problem, with the associated rate of suicide amongst those with BD as high as 15%. In addition to the personal burden, BD also has a large economic impact, costing as much as 195 billion USD/annum globally (Merikangas, et al., Arch Gen Psychiatry, 64:543-52 (2007); Crump, et al., JAMA Psychiatry, 70:931-9 (2013); Jamison, J Clin Psychiatry, 61:47-51 (2000); Bessonova, et al., Clinicoecon Outcomes Res.; 12:481-497 (2020)).

Treatment for BD is limited, mainly including pharmaceutical mood stabilizers, antidepressants, and antipsychotic drugs discovered decades ago. Their efficacy in only a subgroup of BD patients highlights the need for development of new drugs based on a molecular understanding of disease etiology (Bavamian, et al., Mol Psychiatry; 20:548 (2015); Bavamian, et al., Mol Psychiatry.; 20:573-84 (2015)).

The neuropsychiatric disorders BD, schizophrenia, and major depression, as well as other neurodevelopmental disorders such as autism spectrum disorder (ASD) and attention deficit hyperactivity disorder (ADHD), substantially overlap in clinical traits. Many BD patients suffer from cognitive deficits and psychotic symptoms qualitatively resembling those of schizophrenia patients, and from depressed mood states resembling those of major depression patients. For this reason, precise diagnosis often requires extensive psychiatric evaluation based on clusters of symptoms, and in some cases even erroneous pharmacological treatment attempts. Although there is a wide consensus for differential brain structural and connectivity impairments, there is little specific evidence describing neuronal substrates and mechanisms differentiating neuropsychiatric disorders at the cellular level (Strakowski, et al., Bipolar Disord. 14:313-25 (2012); Delvecchio, et al., Eur Neuropsychopharmacol.; 22:100-13 (2012); and Delvecchio, et al., Psychol Med.; 43:553-690 (2013)). Consequently, in the absence of more conclusive biological markers, diagnosing is almost exclusively based on psychiatric evaluation (Linden, Neuron; 73:8-22 (2012)).

Despite robust evidence for genetic susceptibility to BD, with heritability estimated as high as 70-80% based on twin studies (Bienvenu et al., (2011) Psychol Med.), only a few genetic susceptibility factors have been identified over decades of research, with little evidence for BD-specific risk genes (Craddock, et al., Lancet, 381:1654-62 (2013); McGuffin, et al., Arch Gen Psychiatry; 60:497-502 (2003); Kieseppa et al., Am J Psychiatry, 161:1814-21 (2004); Craddock et al., Trends Genet, 25:99-105 (2009); and Edvardsen, et al., J Affect Disord, 106:229-40. (2008)). Genome-wide association studies (GWAS) show substantial commonalities in risk loci for the major psychiatric disorders, especially between BD and schizophrenia, suggesting they overlap not only in clinical symptoms but also in their contributing genetic factors. (O'Donovan, et al., Nat Med., 22:1214-9 (2016); Lee, et al. Nat Genet., 45:984-94 (2013); Purcell, et al., Nature, 460:748-52 (2009); Gandal, et al., Science, 359:693-7 (2018); and Lichtenstein, et al., Lancet, 373:234-9 (2009)).

A handful of common genetic variants, identified by GWAS as robustly associated with BD and replicated across independent studies, are single-nucleotide polymorphisms (SNPs) in the genes CACNA1C, ANK3, ODZ4, SYNE1, and TRANK1. Some of the identified loci show penetrance for two or more of the neuropsychiatric disorders, underlining a shared genetic etiology (Psychiatric GCBDWG., Nat Genet., 43:977-83 (2011); Heyes, et al., Prog Neurobiol., 134:36-54 (2015); Iqbal, et al., Hum Mol Genet., 22:1960-70 (2013); Forstner, et al., PLoS ONE, 12:e0171595 (2017)). The largest GWAS to date for identifying risk loci for neuropsychiatric disorders including BD, schizophrenia, and major depression, identified the region encompassing SYNE1 as the strongest BD association locus in the genome (Psychiatric GWAS Consortium BD Working Group. Nature Genetics 2011). Meta-analyses included in the study identified SNPs in SYNE1 with genome-wide statistically significant association to BD at P=4.27×10⁻⁹ (Psychiatric GCBDWG., Nat Genet., 43:977-83 (2011); Green, et al., Mol Psychiatry, 18:614-7 (2013); Green, et al., Mol Psychiatry, 18:1302-7 (2013); Cross-Disorder Group of the Psychiatric Genomics C., Lancet, 381:1371-9 (2013); Xu, et al., BMC Med Genet., 15:2 (2014)).

Increasing evidence, mainly from genetic and pharmacological studies, has implicated abnormal glutamatergic neurotransmission and synaptic plasticity in the etiology of BD (Sanacora, et al., Nat Rev Drug Discov., 7:426-37 (2008); Nurnberger, et al., JAMA Psychiatry.; 71:657-64 (2014); and Machado-Vieira, et al., Pharmacol Biochem Behav., 100:678-87 (2012)). Notably, SNPs in GRIA2, which encodes the GluA2 subunit of AMPARs, have been associated with time to recurrence of mood episodes in BD patients (Perlis, et al., Am J Psychiatry, 166:718-25 (2009)). Studies have also shown differences in glutamate levels as well as glutamate receptor expression or function between individuals with mood disorders and control subjects (Scarr, et al., Bipolar Disord., 5:257-64 (2003); McCullumsmith, et al., Brain Res., 1127:108-18 (2007); Nudmamud-Thanoi, et al., Neurosci Lett., 372:173-7 (2004); Meador-Woodruff, et al., Brain Res Bul., 5:631-40 (2001); Beneyto, et al., Synapse, 60:585-98 (2006)). While SYNE1 is a large gene encoding multiple transcripts, the SNPS conferring risk to BD are close to an activity-regulated transcript encoding the CPG2 protein (Loebrich, et al., Molecular and Cellular Neuroscience, 71:46-55 (2016)). CPG2 is a localized to the endocytic zone near glutamatergic synapses and plays a role in internalization of synaptic glutamate receptors (Cottrell, et al., Neuron, 44:677-90 (2004)). Samples from post-mortem brains show that CPG2 protein levels are significantly lower in BD patients as compared to control subjects, and that this is not the case for patients with Schizophrenia or major depression (Rathje M et al., Mol Psychiatry 26, 508-523 (2021)). However, current methodologies lack sufficient genetic resolution and statistical power to identify and confirm the link between BD and a particular gene or molecule.

There is a need for developing biomarkers for the diagnosis of BD amongst subjects suspected of having, or at risk of developing BD, that are distinct for BD and not other mood disorders with clinical symptom overlap. There is also a need for developing efficient assays that can assess the likelihood of successful treatment of subjects with BD through one or more treatment regimens. Finally, there is a need for improved treatment regimens for BD, and for monitoring the success of treatments at the molecular level.

Therefore, it is an object of the invention to provide assays for the accurate diagnosis and for predicting treatment efficacy of bipolar disorder amongst subjects suspected of having, or at risk of developing bipolar disorder.

It is another object of the invention to provide high-throughput methods to screen and select active agents effective as potential therapeutic agents for treating bipolar disorder.

It is a further object of the invention to provide high-throughput methods to screen and select individuals for likelihood of successful treatment with one or more therapeutic regimens, based on their genetic sequence.

It is a further object of the invention to provide personalized treatment regimens for bipolar disorder, and for predicting their success at the molecular level.

### SUMMARY OF THE INVENTION

The CPG2 promoter region extends approximately 800 base pairs upstream of the transcription start site, with a second promoter region extending downstream into intron 16. A 200 bp section of the upstream promoter region of the CPG2 gene is responsive to Lithium at doses comparable to patient treatment for BD. Compositions and methods for the detection of polymorphisms within the 200 bp section of the CPG2 promoter region that confer risk for BD have been developed.

Methods for diagnosing bipolar disorder in a subject include measuring the activity of the promoter of the candidate plasticity gene (CPG2) from the subject and selecting the subject as having bipolar disorder or as being at risk of bipolar disorder when the activity of the CPG2 promoter from the subject is significantly less than that of a control promoter. A preferred CPG2 promoter is a nucleic acid sequence spanning 800 base pairs upstream of the transcription start site of CPG2. In some embodiments, the methods also assess the effect of one or more active agents upon the activity of the CPG2 promoter from the subject, wherein the effect of the active agent is determined by comparison with the activity of the CPG2 promoter in the absence of the active agent.

The methods typically employ in vitro transcriptional assays to identify the effect of single nucleotide polymorphisms (SNPs) within the promoter region of the CPG2 gene upon the expression of the CPG2 protein. Compositions and methods for detecting BD in a subject are provided. The methods identify subjects having BD, or at risk of BD, identify potential therapeutic agents and predict effective treatment regimens for prophylactic and therapeutic applications.

Typically, assessing transcription of CPG2 in the subject, or assessing transcription of CPG2 in the presence of one or more active agents, or both, includes an in vitro transcription assay. An exemplary in vitro transcription assay includes one or more steps of (i) obtaining genomic DNA including the promoter of CPG2 from the subject; (ii) sequencing the CPG2 promoter region from the subject, or alternatively instead of steps (i) and (ii) identifying the CPG2 promoter sequence from individual subjects contained in a genomic database; (iii) mapping single nucleotide polymorphisms in the CPG2 promoter sequence of individual subjects; (iv) introducing the identified polymorphisms into the CPG2 promoter within a reporter construct expressing a reporter protein controlled by the CPG2 promoter; (v) expressing the CPG2 reporter constructs with the introduced polymorphisms in one or more cell lines; and (vi) quantitating the expressed reporter protein to assess the activity of the CPG2 promoter from the subject by reference to a control.

Typically, genomic DNA is isolated from a biological sample obtained from the subject. In some embodiments, the promoter of CPG2 includes a nucleic acid of about 200 base pairs in size, preferably, between about 800 bp and about 600 bp upstream of the transcription start site of CPG2. In other embodiments, the promoter of CPG2 includes a nucleic acid sequence from between greater than 1 base pair and 1,000 base pairs, inclusive, upstream of the transcription start site of CPG2. In preferred embodiments, the promoter of CPG2 includes a nucleic acid sequence having one or more single nucleotide polymorphisms (SNPs) associated with bipolar disorder. Exemplary SNPs include one or more selected from rs118135299, rs924872285, rs4530871, and rs4523096. In some embodiments, the control includes one or more expression products regulated by the transcriptional start sequence of CPG2 without single nucleotide polymorphisms (SNPs) associated with bipolar disorder. Typically, a reporter protein under the transcriptional control of the CPG2 promoter are expressed in cultured cortical neuron cells. In some embodiments, the expressed reporter protein includes a luciferase protein, and quantitation includes a luciferase reporter assay. Suitable luciferase reporter assays include a Single Luciferase Reporter Assay, a Dual Non-Secreted Luciferase Reporter Assay, a Dual Secreted Luciferase Reporter Assay, a Real Time Luciferase Reporter Assay, and a Multicolor Luciferase Reporter Assay.

In some embodiments, the methods include one or more steps of selecting one or more active agents as a potential therapeutic agent when the activity of the CPG2 promoter is increased in the presence of the active agent. For example, in some embodiments, an agent is selected as a potential therapeutic agent when the activity of the CPG2 promoter is increased to a level equivalent to that of a control. A preferred active agent is lithium, or a lithium salt.

In some embodiments, the methods include one or more steps of (a) selecting a subject having bipolar disorder for treatment with a therapeutic agent, including assessing transcription of the candidate plasticity gene 2 (CPG2) from the subject both in the presence and in the absence of the therapeutic agent; and (b) selecting the subject for treatment for bipolar disorder with the therapeutic agent when the activity of the CPG2 promoter is increased in the presence of the therapeutic agent. In a preferred embodiment, the therapeutic agent is lithium, or a salt thereof. In particular embodiments, the methods also include the step of (c) selecting the optimal dose of the therapeutic agent. For example, in some embodiments, the methods titrate the amount of the active agent to determine the amount of the agent required to increase the activity of the CPG2 promoter, optionally, the methods titrate the amount of the active agent effective to increase the activity of the CPG2 promoter from the subject equivalent to that of a normal control CPG2 in the absence of the active agent.

In some embodiments, the methods screen new therapeutic agents suitable for treating patients with bipolar disorder. In preferred embodiments, the patients are those unresponsive to lithium.

Typically, the subject is an individual who is suspected of having Bipolar I disorder, Bipolar II disorder, or Cyclothymic disorder. In some embodiments, the subject has one or more psychiatric symptoms selected from hallucinations, suicidality, cognitive deficits, mania, or hypomania (flight of ideas, elevated mood, and pressured speech), depressed mood, concentration problems, anxious distress, melancholy, and psychosis. In other embodiments, the subject does not have any psychiatric symptoms of BD. In some embodiments, the subject is suspected of having, and/or has a family or personal history of mood disorders, such as but not restricted to, schizophrenia or major depression.

Kits for identifying bipolar disorder in a subject are also provided. Typically, kits include one or more of (i) reagents for assessing the activity of the promoter of the candidate plasticity gene 2 (CPG2) in a subject; (ii) a control sample, including one or more expression vectors configured to express a reporter protein regulated by the CPG2 promoter without single nucleotide polymorphisms associated with bipolar disorder; and (iii) instructions for assessing the activity of the promoter of candidate plasticity gene 2 (CPG2) in a subject, where the subject is selected as having bipolar disorder or being at risk of bipolar disorder when the subject has reduced activity of the CPG2 promoter as compared to a control. In some embodiments, the kits also include (iv) one or more active agents; and (v) instructions for assessing the activity of the CPG2 promoter in the presence of one or more active agents, where the activity of the CPG2 promoter in the presence of the active agent is assessed by comparison with the activity of the CPG2 promoter in the absence of the active agent. In some embodiments, kits also include lithium, or a salt thereof.

### BRIEF DESCRIPTION OF THE DRAWINGS

FIG. 1 is a schematic representation of the 145 exons of the human SYNE1 gene, showing the location of the single nucleotide polymorphism (SNPs) linked with BD in a Genome Wide Association Study for identifying risk genes for neuropsychiatric disorders. The line at top shows the reverse strand of human chromosome 6 spanning the SYNE1 locus. Small vertical lines represent exons. Long vertical dashed lines mark sites of disease-associated genetic variations. A star marks the position of the lead SNP identifying SYNE1 as a risk locus for BD. Other lines show representative SYNE1 transcripts annotated from the UCSC Genome database. The BD association signal within SYNE1 maps near the transcription start site for candidate plasticity gene 2 (CPG2). Lines show four transcripts that span in part the region homologous to rat Cpg2 which is the bar including a star at the top.

FIG. 2 is a schematic representation of truncations that were introduced into a WT CPG2 promoter used for luciferase assays. The expression level, indicated by Luciferase activity (−, +, ++, +++, ++++ and +++++, respectively) is indicated for each fragment (at right), with the seven most highly-expressed fragments (−1.0 kb, −0.8 kb, −0.6 kb, −0.4 kb, −0.2 kb, −0.2 kb i16 and 5′UTR i16), as well as the relative orientation of the 200 bp section in the putative CPG2 promoter (in −0.8 kb fragment) that is responsive to Li treatment, are indicated.

FIG. 3 is a bar graph of relative luciferase activity (0-80) for each of the seven most highly expressed fragments (−1.0 kb, −0.8 kb, −0.6 kb, −0.4 kb, −0.2 kb, −0.2 kb i16 and 5′UTR i16, respectively), from a balanced group of male and female samples, treated with/without Lithium (0 mM and 2 mM Lithium, respectively). LiCl treatment (2 mM, 24 hrs) impacts gene expression driven by −0.8 kb promoter region, but not other regions. SV40 is used as a control promoter.

FIG. 4 is a bar graph of relative luciferase activity (0-80) for the −0.8 kb wild-type (control) and the mutant −0.8 kb (rs118135299, SNP299). Expression data from the mutant −0.8 kb SNP299 was compared with data from the control −0.8 kb fragment, taken from FIG. 3. The mutant −0.8 kb (SNP299) blunted the LiCl influence on CPG2 transcription.

### DETAILED DESCRIPTION OF THE INVENTION

### I. Definitions

The term “small molecule,” generally refers to an organic molecule that is less than about 2,000 g/mol in molecular weight, less than about 1,500 g/mol, less than about 1,000 g/mol, less than about 800 g/mol, or less than about 500 g/mol. Small molecules are non-polymeric and/or non-oligomeric.

The term “carrier” refers to an organic or inorganic ingredient, natural or synthetic, with which the active ingredient is combined to facilitate the application.

The term “pharmaceutically acceptable” describes a material that is not biologically or otherwise undesirable. The term refers to those compounds, materials, compositions, and/or dosage forms which are, within the scope of sound medical judgment, suitable for use in contact with the tissues, organs, and/or bodily fluids of a subject without excessive toxicity, irritation, allergic response, or other problems or complications commensurate with a reasonable benefit/risk ratio. Such materials can be administered to a subject along with the selected compound without causing any undesirable biological effects or interacting in a deleterious manner with any of the other components of the pharmaceutical composition in which it is contained.

The term “pharmaceutically-acceptable carrier” means one or more compatible solid or liquid fillers, dilutants or encapsulating substances which are suitable for administration to a human or other vertebrate animal.

The terms “treatment” or “treating” means to administer a composition to a subject or a system with an undesired condition (e.g., an infectious disease, cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The terms “effective amount” or “therapeutically effective amount” are used interchangeably and mean a quantity sufficient to alleviate or ameliorate one or more symptoms of a disorder, disease, or condition being treated or to otherwise provide a desired pharmacologic and/or physiologic effect. Such amelioration only requires a reduction or alteration, not necessarily elimination. The precise quantity will vary according to a variety of factors such as subject-dependent variables (e.g., age, health, weight, etc.), the disease or disorder being treated, the disease stage, as well as the route of administration, and the pharmacokinetics and pharmacodynamics of the agent being administered.

The term “detect,” “detecting,” “determine” or “determining” generally refers to obtaining information. Detecting or determining can utilize any of a variety of techniques available to those skilled in the art, including for example specific techniques explicitly referred to herein. Detecting or determining may involve manipulation of a physical sample, consideration and/or manipulation of data or information, for example utilizing a computer or other processing unit adapted to perform a relevant analysis, and/or receiving relevant information and/or materials from a source. Detecting or determining may also mean comparing an obtained value to a known value, such as a known test value, a known control value, or a threshold value. Detecting or determining may also mean forming a conclusion based on the difference between the obtained value and the known value.

The term “diagnosing” refers to identifying the nature of a disease or condition that a subject may be suffering from. The term “diagnosis” refers to the determination and/or conclusion that a subject suffers from a particular disease or condition. The term “diagnosing” may denote the disease's identification (e.g., by an authorized physician or a test approved from a health care authority).

The term “subject” means any individual, organism, or entity. The subject can be a vertebrate, for example, a mammal. Thus, the subject can be a human. The term does not denote a particular age or sex. Thus, adult, and newborn subjects, as well as fetuses, whether male or female, are intended to be covered. The subject may be healthy or suffering from or susceptible to a disease, disorder, or condition. A patient refers to a subject afflicted with a disease or disorder. The term “patient” includes human and veterinary subjects.

Recitation of ranges of values herein are merely intended to serve as a shorthand method of referring individually to each separate value falling within the range, unless otherwise indicated herein, and each separate value is incorporated into the specification as if it were individually recited herein.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “vector” refers to a replicon, such as a plasmid, phage, or cosmid, into which another DNA segment may be inserted to bring about the replication of the inserted segment. The vectors can be expression vectors.

The term “expression vector” refers to a vector that includes one or more expression control sequences.

The terms “promoter” and “transcription control sequence” refer to a DNA sequence that controls and regulates the transcription and/or translation of another DNA sequence. Control sequences that are suitable for prokaryotes, for example, include a promoter, optionally an operator sequence and/or a ribosome binding site. Eukaryotic cells are known to utilize promoters, polyadenylation signals, and enhancers.

The terms “transformed,” “transgenic,” “transfected” and “recombinant” refer to a host organism into which a heterologous nucleic acid molecule has been introduced. The nucleic acid molecule can be stably integrated into the genome of the host, or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism e.g., a bacterium or plant, or cells, which does not contain the heterologous nucleic acid molecule.

The terms “candidate plasticity gene 2” or “cpg2” are used interchangeably to refer a transcript that is a product of the human SYNE1 gene, a large gene encoding multiple proteins with different functions. CPG2 refers to the protein encoded by this transcript. The cpg2 expression pattern, activity-dependent regulation, and sub-cellular localization of its protein product are all consistent with a role in synaptic plasticity (Cottrell et al., Neuron 2004). The CPG2 protein is localized to an endocytic zone adjacent to excitatory synapses in dendritic spines. At this site, CPG2 plays a role in constitutive as well as activity-dependent endocytosis of synaptic glutamate receptors (Cottrell et al., Neuron 2004). At the synapse, clathrin mediated endocytosis (CME) of AMPA-type glutamate receptors (AMPARs) from the postsynaptic membrane is thought to be the substrate for various forms of plasticity, including long-term depression (LTD).

### II. Methods

Methods for diagnosing and evaluating treatment efficacy for neuropsychiatric disorders, including genetically determined mood disorders such as bipolar disorder (BD) based on in vitro transcriptional assays have been established.

In some embodiments, the methods provide a diagnosis for a neuropsychiatric disease, such as a heritable mood disease or disorder, based on the detection of one or more molecular markers in a sample of genomic DNA from a subject. In other embodiments, the methods identify one or more substances that are active in altering the amount or presence of one or more molecular markers associate with a neuropsychiatric disease. In further embodiments, the methods identify a subject having a neuropsychiatric disease as being a candidate for effective treatment with one or more substances that are active in altering the amount or presence of one or more molecular markers associated with the neuropsychiatric disease.

In particular, the methods exploit the discovery that glutamate receptor recycling, the candidate plasticity gene 2 (CPG2), and control of its levels by specific regulatory regions of the CPG2 promoter, are associated with bipolar disorder (BD). For example, in some embodiments, methods for identifying bipolar disorder in a subject include assessing transcription of CPG2 in the subject, where the subject is selected as having bipolar disorder or being at risk of bipolar disorder when the subject has reduced transcription of CPG2 as compared to a control. In some embodiments, finding that a subject's genomic DNA sequence contains SNPs previously shown to attenuate CPG2 transcription is sufficient to diagnose BD. In other embodiments, the methods include one or more steps to investigate the activity or applicability of one or more test compounds to treat or alleviate one or more symptoms of BD. In further embodiments, the methods predict optimal treatment regimens for a subject for BD based on the ability of an active agent to enhance transcription of CPG2 in an in vitro assay.

A. Bipolar Disorders (BD)

Bipolar disorder (BD), previously known as manic depression, is a debilitating and heritable mood disorder characterized by recurrent episodes of mania and depression. Drug treatment is often unsuccessful, and full recovery between episodes is not achieved in all patients. As a consequence, BD is a leading cause of disability, loss of life through high suicide rates and contributes to the emotional and financial burden for families and society. Growing evidence suggests that the glutamatergic system is central to the neurobiology of mood disorder.

Despite robust evidence for genetic susceptibility to BD, with heritability estimated as high as 70-80% based on twin studies, only a few genetic susceptibility factors have been identified over decades of research (Bienvenu et al. (2011) Psychol Med.). Consequently, in the absence of more conclusive biological markers, diagnosis of BD in the clinic is almost exclusively based on psychiatric evaluation. As a result, many other neuropsychiatric diseases and behavior disorders are commonly confused with BD, including Schizophrenia, Alzheimer's Disease, Panic Disorder, Major Depression and Generalized Anxiety, Cocaine disorder, Anorexia nervosa, Alcoholism, Sedative Drug Disorder, Cannabis use disorder, and Stimulant use disorder.

All of the methods can include one or more steps of selecting a subject. In some embodiments, a subject is an individual who is suspected of having bipolar disorder (BD). In some embodiments, a subject is an individual with BD, or who is at risk of developing BD. In some embodiments, a subject with BD, or who is at risk of developing BD but does not have one or more other neuropsychiatric diseases and behavior disorders including Schizophrenia, Alzheimer's Disease, Panic Disorder and Major Depression. In particular embodiments, a subject suspected of having BD has a family or personal history of BD and/or other mood disorders. In other embodiments, a subject suspected of having BD has previously been treated with one or more active agents effective against Schizophrenia, Alzheimer's Disease, Panic Disorder, and Major Depression.

There are several types of bipolar and related disorders, including Bipolar I disorder, Bipolar II disorder, Cyclothymic disorder, and other types of bipolar disorder. Bipolar I disorder is characterized by unpredictable changes in mood and behavior, resulting in significant distress and difficulty in life. In some cases, mania may trigger a break from reality (psychosis). Bipolar II disorder is characterized by at least one major depressive episode and at least one hypomanic episode, without a manic episode. Bipolar II disorder is not a milder form of bipolar I disorder, but a separate diagnosis. While the manic episodes of bipolar I disorder can be severe and dangerous, individuals with bipolar II disorder can be depressed for longer periods, which can cause significant impairment. Cyclothymic disorder is characterized by at least two years in adults, or one year in children and teenagers, of many periods of hypomania symptoms and periods of depressive symptoms (though less severe than major depression). Therefore, in some embodiments, a subject is an individual who is suspected of having Bipolar I disorder, Bipolar II disorder, or Cyclothymic disorder.

1. Symptoms of BD

Typical psychiatric symptoms of bipolar disorders are similar to those of other psychiatric diseases and include hallucinations, suicidality, cognitive deficits, mania and hypomania (flight of ideas, elevated mood and pressured speech), depressed mood, concentration problems, anxious distress, melancholy, and psychosis. Therefore, in some embodiments, a subject is an individual may or may not be diagnosed with BD, but who has one or more symptoms of BD, including hallucinations, suicidality, cognitive deficits, mania, and hypomania (flight of ideas, elevated mood and pressured speech), depressed mood, concentration problems, anxious distress, melancholy, and psychosis.

Psychiatric symptoms of BD typically manifest in mania or hypomania and depression. Mania and hypomania are two distinct types of episodes with corresponding symptoms. Mania is more severe than hypomania and causes more noticeable problems at work, school, and social activities, as well as relationship difficulties. Mania may also trigger a break from reality (psychosis) and require hospitalization. The timing of symptoms may include diagnostic labels such as mixed or rapid cycling. In addition, bipolar symptoms may occur during pregnancy, postpartum, or change with the seasons. Both a manic and a hypomanic episode include three or more symptoms of abnormally upbeat, jumpy or wired, increased activity, energy or agitation, exaggerated sense of well-being and self-confidence (euphoria), decreased need for sleep, unusual talkativeness, racing thoughts, distractibility, and poor decision-making (for example, going on buying sprees, and taking risks or making foolish investments). Therefore, in some embodiments, a subject is an individual who has or is suspected of having one or more of a manic, a hypomanic episode, or a major depression episode.

B. Methods for Diagnosing Bipolar Disorders

Methods for screening and/or diagnosing BD at a significantly high level of specificity and sensitivity are provided. The methods identify a subject who has BD, is at risk of developing BD, or who is a carrier for BD. Typically, this is based on determining that the subject has one or more genetic variants that are known or are determined by the methods provided to increase risk for BD.

1. Molecular Markers of Bipolar Disorders

In some embodiments, the methods detect one or more molecular markers associated with BD. Therefore, the systems and methods are useful to detect or diagnose BD in a subject. Preferred molecular markers include genetic variants associated with BD. After determining the presence of one or more genetic variants, the identified variants can be compared to a database to determine whether there is any known association of the one or more variants with a disease. If one or more of the identified genetic variants is known to indicate a risk of developing BD, or to be causative of, contributive to, or associated with BD, then the subject is diagnosed accordingly. For example, in some embodiments, molecular markers of BD are identified by evaluation against a database. Database records include extensive phenotypic descriptions, biochemical and hematological effects, associated pathology, and ethnic occurrence, accompanied by mutation frequencies and references. Suitable databases include, without limitation, the IthaGenes database, the Human Gene Mutation Database (HMGD), ClinVar database, GnomAD database, and the Single Nucleotide Polymorphism Database (dbSNP).

In some embodiments, a subject is diagnosed as having BD or being a carrier thereof when the subject is identified as having one or more candidate plasticity gene 2 (CPG2) variants associated with reduced expression of CPG2. For example, if one or more single nucleotide polymorphisms in the promoter region of the CPG2 variant are detected (and optionally validated) in the subject's sample, the subject can be diagnosed with BD.

Preferably, methods provide a library of SNPs from data collected from patients over time. Therefore, in some embodiments, the diagnosis is carried out based on screening SNPs against the library of available SNPs without any in vitro testing.

a. Candidate Plasticity Gene 2 (CPG2)

It has been established that the bipolar disorder (BD) association signal within SYNE1 maps near the transcription start site for the candidate plasticity gene 2, cpg2 transcript.

Both genetic and environmental factors have been implicated in the etiology of BD. Genome-wide association studies (GWAS) of neuropsychiatric disorders have identified a risk locus for BD within the SYNE1 gene, a large gene encoding multiple proteins. The BD association signal spans, almost exclusively, the part of SYNE1 encoding CPG2, a brain-specific protein localized to excitatory postsynaptic sites, where it regulates glutamate receptor internalization. CPG2 is present within the human SYNE1 gene and plays a role in regulating excitatory synaptic strength.

The cpg2 transcript is a product of the SYNE1 gene, a large gene encoding multiple proteins with different functions. Candidate Plasticity Gene 2 (CPG2) encodes a protein that localizes to the post-synaptic spines of excitatory neurons where it regulates the internalization of glutamate receptors. CPG2 has a modular structure with multiple protein interaction domains suggesting it functions as a large adaptor complex that can mediate the interaction of varied synaptic components involved in regulation of glutamate receptor cycling at the synapse. At the synapse, clathrin-mediated endocytosis (CME) of AMPA-type glutamate receptors (AMPARs) from the postsynaptic membrane is thought to be the substrate for various forms of plasticity, including long-term depression (LTD). Removal of CPG2 increases synaptic glutamate receptor levels, which results in dysregulated synapses, one of the underlying mechanisms described for bipolar disorder.

Therefore, methods for detecting BD in a subject include assessing the activity of the CPG2 promoter and/or the levels of expressed CPG2 protein in the subject.

It has been shown that CPG2 protein levels are significantly decreased in postmortem brain tissue from BD patients, as compared to control subjects, as well as schizophrenia and depression patients (Rathje M, et al., Molecular Psychiatry, 26, 508-523 (2021). Genetic variants within the postmortem brains that map to the CPG2 promoter region have been identified, and it has been shown that they negatively affect gene expression. Missense single nucleotide polymorphisms (SNPs) have also been identified in CPG2 coding regions that affect CPG2 expression, localization, and synaptic function. Accordingly, genetic variation in the CPG2 region of SYNE1 is linked with a mechanism for glutamatergic synapse dysfunction that could underlie susceptibility to BD in some individuals.

b. CPG2 Promoter

Several Single Nucleotide Polymorphisms (SNPs) have been identified within the putative promoter region of the human CPG2 that have been associated with bipolar disorder, including rs924872285, rs4530871, and rs4523096. These polymorphisms are typically more frequently present in the putative CPG2 promoter region of genomic DNA samples obtained from BD patients. Since the promoter region is critical for the correct transcription and expression rate of the CPG2, methods for assessing BD status and/or risk of BD and/or suitability of therapeutic agents for treatment of BD in a subject in need thereof include assessing the activity of the CPG2 promoter. Preferred methods for assessing the activity of the CPG2 promoter and/or the CPG2 expressed protein in the subject include using a cell-based in vitro transcription assay. Therefore, methods for detecting BD typically identify one or more genetic determinants of BD within the promoter region of CPG2 of a subject. In particular, the methods assess the activity of the promoter of CPG2.

In some embodiments, the methods extract genomic DNA from a subject and characterize the sequence of the promoter of the CPG2 from the genomic DNA of the subject. In other embodiments, the methods characterize a specific fragment of the promoter of CPG2. Exemplary fragments include 10-1000 base pairs (bp), such as 10 bp, 20, 30, 40, 50, 60, 70, 80, 90, 100, 150, 200,250, 300, 400, 500, 600, 700, 800, 900 or 1,000 bp. Fragments are typically located 0-1,000 bp upstream of the transcription start site of CPG2, upstream of the 5′UTR region of CPG2, or can be located in intronic regions downstream of the CPG2 transcription start site. Therefore, a fragment of the CPG2 promoter includes a nucleic acid sequence of 10-1,000 contiguous residues isolated from a specific location from spanning between about 1 bp to about 1,000 bp upstream of the transcription start site of CPG2 or upstream of the 5′UTR region of CPG2, or in intronic regions downstream of the CPG2 transcription start site. Exemplary fragments include 0-1,000 bp upstream of the transcription start site of CPG2, 0-800 bp, 0-600 bp, 0-400 bp, 0-200 bp, 200-1000 bp, 400-1000 bp, 600-1000 bp, and 800-1000 bp upstream of the transcription start site of CPG2, 200-800 bp, 400-800 bp, and 600-800 bp upstream of the transcription start site of CPG2, and 200-600 bp, 400-600 bp, and 200-400 bp upstream of the transcription start site of CPG2. If the activity of the fragment of the CPG2 promoter is reduced, or if the expression or level or activity of a CPG2 promoter-expressed protein is reduced relative to the expression of a protein by a healthy control promoter, the subject is identified as having bipolar disorder. The methods optionally include one or more steps for assessing the responsiveness of the CPG2 promoter and/or the CPG2 promoter-expressed protein to one or more active agents. In particular, the methods assess the responsiveness of a fragment of the CPG2 promoter spanning 200 base pairs from between 800 bp and 600 bp upstream of the transcription start site of CPG2 to one or more active agents.

2. In Vitro Transcription Assay

In a preferred embodiment, the methods employ an in vitro transcription assay to identify one or more genetic determinants of BD in a subject. Methods to assess the activity of the CPG2 promoter and/or the level of expressed CPG2 protein in the subject include one or more steps of performing an in vitro transcription assay to identify one or more genetic determinants of BD in a subject. In an exemplary embodiment, the in vitro transcription assay includes one or more steps of
(i) obtaining genomic DNA including the promoter region of CPG2 from the subject, or obtaining DNA sequence information from the CPG2 region from a patient database that has genomic data of the subject;
(ii) introducing SNPs identified in the CPG2 promoter region from the subject's DNA sequence into an expression vector configured to express a reporter protein controlled by the CPG2 promoter;
(iii) expressing the reporter protein controlled by the CPG2 promoter with or without the identified SNPs in primary neuronal cultures or a cell line; and
(iv) quantitating the expressed reporter protein to assess the activity of the CPG2 promoter containing SNPs from the subject by reference to a control.

An exemplary nucleic acid sequence of a region of the CPG2 promoter that is responsive to LiCl spans 200 base pairs from between 800 bp and 600 bp upstream of the transcription start site of CPG2 is set forth below:

(SEQ ID NO: 1)
agttttcact tgtcccacca agtgtgtctc ttgattttag
gctggttttg ctactaattt ctctaatttg ctaaatttta
gtatgagata aaaatattaa ttttaatgta ttattttggt
ggtctaatat gtcttacttt ttcaatgagt gacacatttt
taagtactgt atttattctg caatcaagac acaaactaag.

The methods introduce the nucleic acid sequence of a region of the CPG2 promoter spanning 200 base pairs from between 800 bp and 600 bp upstream of the transcription start site of CPG2 into an expression vector, to assess the rate and/or amount of expression of one or more expression products under the control of the CPG2 promoter region, in the presence of LiCl or other pharmaceutical agents. Therefore, the methods typically include one or more steps to introduce the nucleic acid sequence of a region of the CPG2 promoter into an expression vector. In some embodiments, the methods sequence the CPG2 promoter region of genomic DNA from the subject to identify SNPs within the CPG2 promoter. In other embodiments, the methods analyze DNA databases including subject genomic sequences to identify SNPs within the CPG2 promoter. The methods then include one or more steps to introduce any SNPs present within the subject's DNA as point mutations into a control promoter, for example, a promoter including the nucleic acid sequence of SEQ ID NO:1. In other embodiments, the promoter region of the CPG2 gene is isolated from the genomic DNA of a subject and cloned into an expression vector, for example, a vector including a reporter gene. Methods of obtaining and/or sequencing genomic DNA from a subject by isolating nucleic acids from a biological sample obtained from the subject are known in the art. Biological samples can be any sample of tissue, bodily fluid, cells or other material that contains genomic DNA. The vector is transfected, optionally together with one or more control vectors, into a target cell that is cultured in vitro. Exemplary target cells include neuronal cells, such as cultured cortical neurons.

In preferred embodiments, genomic DNA sequence of the CPG2 region is obtained from a patient database that already has the genomic data of the subject. Thus, when patient's genomic data are readily available, it involves identifying the region of interest (e.g., CPG2 promoter) and examine the region for SNPs.

In a preferred embodiment, the in vitro transcription assay employs a reporter system to rapidly and effectively identify transcription activity of a fragment of the CPG2 promoter spanning any part of the region up to 1 kilobase upstream of the transcription start site including the 5′ untranslated region. An exemplary reporter system is a bioluminescence reporter system. In a typical embodiment, the promoter region of the nucleic acid sequence of the CPG2 gene is isolated from the genomic DNA of a subject and introduced into an expression vector, for example, a vector including a bioluminescent reporter gene.

In an exemplary assay, cortical neurons from rat embryos are cultured in vitro. At DIV 8, neurons are transfected with plasmids containing various CPG2 promoter regions cloned in front of the firefly Luciferase reporter gene. All neurons are also transfected with another plasmid that allows expression of Renilla luciferase which serves as an internal control. At a suitable stage, for example, division number 14 (DIV 14), the cells are lysed to collect the proteins, and gene expression is quantified based on bioluminescent signaling generated right after adding the substrates for the luciferase.

a. Bioluminescence Cell-Based Assays

In a preferred embodiment, the in vitro transcription assay employs a bioluminescence reporter system to rapidly and effectively measure transcriptional level of the CPG2 gene isolated from a subject. In some embodiments, the methods employ an in vitro transcription assay to identify one or more genetic determinants of a mood disorder in a subject.

Bioluminescence is a unique light source based on the luciferin-luciferase reaction, and the luciferases are good reporter enzymes. Bioluminescence reporters are widely used in various aspects of biological functions, such as gene expression, post-translational modification, and protein-protein interaction in cell-based assays. Advanced luciferase technologies also permit the quantitative visualization of gene expression at single-cell resolution by imaging its luminescence in real-time using a highly sensitive charged-coupled device (CCD) camera. Although fluorescence imaging techniques that use fluorescent proteins (e.g., green fluorescent protein (GFP) and its derivatives) as probes have greatly contributed to the advancement of cell biology studies, bioluminescence imaging is emerging as a new and sensitive approach for understanding cell physiology.

Therefore, methods for detecting neuropsychiatric disorders in a subject include assessing the transcriptional activity of the CPG2 gene and/or the CPG2 gene product in the subject using an in vitro transcription assay employing a bioluminescence reporter system. An exemplary bioluminescence reporter is luciferase.

i. Luciferase Reporter Assay

In preferred embodiments, methods for detecting neuropsychiatric disorders in a subject include an in vitro transcription assay using a luciferase reporter system.

Cell based assay systems using reporter enzymes are used widely to study promoters, interactions between promoters and transcription factors, signal transduction, and other cellular activities. Cell based assays are also applied to drug screening both in vitro and in vivo.

Of the reporter genes known to date, luciferases, enzymes that catalyze bioluminescence reactions, are used most frequently because their sensitivity and linear response range are superior to those of typical reporters, including β-galactosidase, chloramphenicol acetyltransferase, glucuronidase, and green fluorescent protein.

Bioluminescence is a simple reaction that is triggered by the addition of luciferin solution and some cofactors, and the equipment for measuring light intensity is simple because it uses only a photomultiplier or a charge-coupled device (CCD) camera; thus, this assay can be applied to high-throughput screening (HTS). Luciferases are the most suitable reporter enzymes for the quantitative measurement of gene expression.

Commercially available luciferases include those from luminescent beetles, sea pansy, copepods, and ostracods. Commercially available luciferins include only three types: firefly D-luciferin, coelenterazine, and Cypridina luciferin, although dinoflagellate and Latia luciferins have also been identified. In light-emitting reactions, the emission maxima of firefly D-luciferin-type, coelenterazine-type, and Cypridina luciferin-type bioluminescence are found at around 535-630 nm, 460-480 nm, and 460 nm, respectively. These values correspond to the fluorescence emission maxima of their oxyluciferins. The mass of luciferases varies widely (20-62 kDa), and their structures, which originate from phylogenetically distant systems, belong to different superfamilies. Cypridina, Gaussia, and Metridia luciferases (CLuc, GLuc, and MetLuc, respectively) are secreted enzymes. The luciferin-luciferase reaction is triggered by adding luciferin, although the bioluminescence in firefly, click beetle, and railroad worm needs ATP and magnesium ions as cofactors.

(1) Single Luciferase Reporter Assay

In some embodiments, the methods employ a single luciferase reporter assay. All applications of bioluminescence systems are based on the principle of a chemical reaction; that is, the light intensity as the measurable product depends on the amounts of luciferase, luciferin, and cofactor(s). Therefore, in the methods for detecting CPG2 transcription, luciferase is used as a reporter enzyme to estimate gene expression in prokaryotic or eukaryotic cells, because the amount of luciferase correlates with light intensity in the presence of excess luciferin and ATP. Reporter enzyme assays are used widely in promoter analysis.

In an exemplary embodiment, the luciferase gene containing the target promoter region of interest in the plasmid is transfected into target cells, and luciferase-expressing cells are lysed after for an appropriate period allowed for the expression of the proteins, e.g., 7 days. The amount of expressed luciferase protein can be estimated from the light intensity in vitro.

In the transient transfection luciferase assay, the luciferase-expressing cells are first lysed for an appropriate period. Luciferase activity in the cell extracts, measured with a luminometer equipped with a photomultiplier tube, is the most frequently used luciferase assay system because of its high sensitivity and broad linear response (up to 7-8 orders of magnitude). Therefore, in some embodiments, the methods employ a transient transfection luciferase assay system to assess CPG2 gene transcriptional activity, and/or gene expression for diagnostic and drug discovery applications. In some embodiments, the methods normalize the promoter activity using cell number or cellular internal enzyme activity, such as β-galactosidase, β-glucuronidase, chloramphenicol acetyltransferase, thus minimizing inherent experimental variability that can undermine experimental accuracy.

In an exemplary embodiment, methods for assessing the activity of a fragment of the CPG2 promoter spanning 200 base pairs from between 800 bp and 600 bp upstream of the transcription start site of CPG2 from a subject include a single luciferase reporter assay according to one or more steps of (a) preparing a reporter plasmid vector; (b) transfecting the plasmid into primary cultured neurons or a cell line; (c) maintaining the cells to allow expression of the luciferase protein with luciferin to produce light; (d) lysing the luciferase-expressing cells to collect the expressed luciferase protein; and (e) quantifying the amount of expressed luciferase protein to determine the transcriptional activity of the CPG2 gene from the subject.

Typically, the reporter plasmid vector includes the target CPG2 promoter sequence and a luciferase gene sequence. In some embodiments, the assessment of promoter activity is normalized. For example, in some embodiments, the promoter activity is normalized by cell numbers or cellular enzymatic activity.

(2) Dual Non-Secreted Luciferase Reporter Assay

In some embodiments, the methods employ a dual non-secreted luciferase reporter assay. For example, in some embodiments, the methods include an additional luciferase as an internal control reporter (dual-reporter assay), thereby minimizing inherent experimental variability that can undermine experimental accuracy, such as differences in the number and viability of cells used, the efficiency of cell transfection and lysis, and so on. In an exemplary embodiment, firefly luciferase from Photinus pyralis (FLuc) is used as the experimental reporter, and Renilla luciferase is used as the internal control reporter, which connects to a constitutively expressed promoter, such as the herpes simplex virus thymidine kinase (HSV-TK) promoter, cytomegalovirus (CMV) immediate-early promoter, or simian virus 40 (SV40) promoter. In some embodiments, the assay system uses two combinations of reporters, for example, the Dual-Luciferase Reporter Assay System from Promega, in which firefly luciferase and Renilla luciferase are used as the experimental reporter and the internal control reporter, respectively. Therefore, in some embodiments, both luciferase activities are measured sequentially from single extracts based on their bioluminescent substrate specificity. For example, Firefly luciferase activity is measured first by adding firefly D-luciferin, and then Renilla luciferase activity is measured by subsequentially adding coelenterazine (another name of Renilla luciferin) with the concomitant quenching of firefly luciferase luminescence. Typically, firefly luciferase activity is normalized by Renilla luciferase activity as a promoter activity. The dual luciferase assay system makes it possible to perform simultaneous monitoring of gene expression, intracellular detection of bioactive compounds, and high throughput screening (HTS) in vitro.

In an exemplary embodiment, methods for assessing the activity of a fragment of the CPG2 promoter spanning 200 base pairs from between 800 bp and 600 bp upstream of the transcription start site of CPG2 from a subject include a dual non-secreted luciferase reporter assay according to one or more steps of (a) preparing a first reporter plasmid vector including a target CPG2 promoter sequence and a firefly luciferase (FLuc; λmax=560 nm) luciferase gene sequence; and a second reporter plasmid vector including a constitutive promoter sequence (TK promoter) and a Renilla luciferase (RLuc; λmax=480 nm) luciferase gene sequence serving as a control; (b) transfecting the two plasmid vectors into primary cultured neurons or a cell line (c) maintaining the cells to allow expression of both of the FLuc and RLuc luciferase; (d) optionally lysing the luciferase-expressing cells to release the proteins when a transient transfection/non-secreted system is used; and (e) quantifying the amount of each of the expressed FLuc and RLuc luciferase proteins to determine the transcriptional activity of the CPG2 gene from the subject.

In some embodiments, FLuc protein catalyzes a reaction with bettle luciferin to produce green light. In some embodiments, RLuc protein catalyzes a reaction with coelenterazine to produce blue light. For example, in some embodiments, expressed luciferase proteins are collected from lysed cells, and the amounts of FLuc and RLuc proteins are estimated from the light intensity separately. The light intensities indicate the target promoter and control promoter activities in living cells. In this case, the target promoter activity is normalized by control promoter activity. Methods for detecting and normalizing light intensity using a dual non-secreted luciferase reporter assay are known in the art. See, for example, Wu, et al., Biotechniques 42: 290-292 (2007).

(3) Multicolor Luciferase Reporter Assay

In some embodiments, the methods employ a multicolor luciferase reporter assay. The methods employ a multicolor luciferase reporter assay system whereby gene expression, protein-protein interaction, ligand binding, and post-transcriptional modification can be monitored simultaneously in a one-step reaction in single-cell extracts. Therefore, in some embodiments, a multicolor luciferase reporter assay system is employed for diagnostic and drug discovery purposes, both of which require the analysis of a huge number of samples.

A multicolor luciferase reporter assay system saves time and money, reduces the amount of sample needed, and facilitates the interpretation of data. Beetle luciferases emit different colors on reacting with firefly D-luciferin, and each emission can be separated by an optical filter. Currently, beetle luciferases (CBGluc, CBRluc, ELuc, SLR, SLG, and SLO) from several species, which display a wide spectral range, are commercially available.

In some embodiments, methods of using the multicolor luciferase reporter assay system include the green-emitting luciferase from Rhagophthalmus ohbai (SLG; λmax=550 nm) (Ohmiya, et al., Sci Rept Yokosuka City Mus. 7: 31-38 (2000)), the orange-emitting luciferase, which is a point mutant of the green-emitting luciferase (SLO; λmax=580 nm) (Viviani et al., 2001), and the red-emitting luciferase (SLR; λmax=630 nm) from Phrixothrix hirtus (Viviani, et al., Biochemistry 38: 8271-8279 (1999)) are used as the reporters.

In an exemplary embodiment, methods for assessing the activity of a fragment of the CPG2 promoter spanning 200 base pairs from between 800 bp and 600 bp upstream of the transcription start site of CPG2 from a subject include a multicolor luciferase reporter assay according to one or more steps of (a) preparing a first reporter plasmid vector including a first target CPG2 promoter (Promoter 1) and the green-emitting beetle luciferase gene sequence; and a second reporter plasmid vector optionally including a second target promoter sequence (Promoter 2) and the orange-emitting beetle luciferase gene sequence; and a third reporter plasmid vector including a constitutive promoter sequence, and the red-emitting beetle luciferase gene sequence as a control; (b) transfection of the three plasmid vectors into target cells; (c) culturing the cells to express all three of the green-, orange-, and red-emitting luciferase proteins to catalyze a reaction with firefly luciferin to produce green, orange and red light, respectively; (d) optionally lysing the luciferase-expressing cells to release the proteins when a transient transfection/non-secreted system is used; and (e) quantifying the amount of each of the expressed luciferase proteins to determine the transcriptional activity of the CPG2 gene and optionally second target promoter sequence from the subject.

The amounts of expressed luciferase proteins can be estimated from the light intensities, the results indicating the amounts of two target promoters and control promoter activities in living cells. In this case, two target promoter activities (green luciferase for Promoter 1 and orange luciferase for Promoter 2) are normalized by the control promoter activity (red luciferase). Methods for detecting and normalizing light intensity using a multicolor luciferase reporter assay are known in the art. See, for example (see Nakajima, et al., Biotechniques 38:891-894 (2005)).

Since all of the green-, orange-, and red-emitting luciferase proteins emit light with firefly D-luciferin, their individual activities can be detected in a one-step reaction in a single sample using filters. For example, the methods can include one or more steps of (i) measuring total relative light units (F0) in the absence of the filters; (ii) then, measuring the F1 value that passed through the O56 filter; and (iii) measuring the F2 value that passed through the R60 filter.

In an exemplary embodiment, each luciferase activity is calculated using the simultaneous equation by substituting F0, F1, and F2 values: where G, O, and R are the activities of the green-, orange-, and red-emitting luciferases, respectively; κGO56, κOO56, and κRO56 are the transmission coefficients of the green-, orange-, and red-emitting luciferases of the O56 filter, respectively; and κGR60, κOR60, and κRR60 are the transmission coefficients of the green-, orange-, and red-emitting luciferases of the R60 filter, respectively. Using this method, the linear response range of the system exceeds two orders of magnitude, although the low-threshold light intensities require one order of magnitude higher intensity than those estimated in the dual-color luciferase assay system.

(4) Real Time Luciferase Reporter Assay

In some embodiments, the methods employ a real-time luciferase reporter assay.

One advantage of using a luciferase reporter is that the luminescence can be measured quantitatively and longitudinally with real-time resolution in vitro and in vivo, when a bioluminescent substrate is administered into cells (Dothager, et al., Curr. Opin. Biotech. 20:45-53. (2009)). Among the possible luciferase-luciferin reactions, the firefly luciferase and firefly D-luciferin pair is used most widely, because the luminescence generated by the reaction can be quantified precisely, and has an extremely low background, and because firefly D-luciferin is highly stable and easily permeates cells and tissues. Firefly luciferase and D-luciferin are suitable for use as the reporter and substrate, respectively, for noninvasive measurements.

Therefore, in some embodiments, after transfection of a reporter gene vector including a CPG2 promoter from a subject, the number of luciferase-expressing cells in a culture plate is measured with a real time monitoring luminometer equipped with a photomultiplier tube. In some embodiments, a non-invasive multicolor luciferase assay is used in in vitro and in vivo studies of post-translational stabilization, signaling pathways, and protein-protein interactions by using a CCD camera to capture the luciferase emissions.

In an exemplary embodiment, methods for assessing the activity of a fragment of the CPG2 promoter spanning 200 base pairs from between 800 bp and 600 bp upstream of the transcription start site of CPG2 from a subject include a real-time luciferase reporter assay according to one or more steps of (a) preparing a reporter plasmid vector including the target promoter sequence and the luciferase gene sequence; (b) transfection of the plasmid into target cells; (c) culturing the cells to express the luciferase protein with luciferin in the medium to produce light; and (e) quantifying the amount of expressed luciferase protein within the cells to determine the transcriptional activity of the CPG2 gene from the subject.

The amount of expressed luciferase protein can be estimated from the light intensity is measured by a real-time monitoring luminometer. The light intensity indicates the promoter activity in living cells. Methods for detecting and normalizing light intensity using a real-time luciferase reporter assay are known in the art. See, for example, Noguchi, et al., BMC Biotechnol. 8: 40. (2008)).

ii. Luciferases

The luciferase reporter assays can include one or more different luciferases.

Exemplary luciferases that can be used in the described methods are set forth in the Table below:

TABLE 1
Luciferases for use in in vitro transcription assays.

| Organism | Luciferase |
|---|---|
| Photinus pyralis | N. American firefly luciferase |
| Luciola cruciate | Jp firefly (Genji-botaru) luciferase |
| Luciola italic | Italian firefly luciferase |
| Luciola lateralis | Jp firefly (Heike) luciferase |
| Luciola mingrelica | E. European firefly luciferase |
| Photuris pennsylvanica | Pennsylvania firefly luciferase |
| P. plagiophthalamus | Click beetle luciferase |
| Phrixothrix hirtus | Railroad worm luciferase |
| Renilla reniformis | Renilla luciferase |
| Rluc8 (mut of Renilla luciferase) | |
| Green Renilla luciferase | |
| Gaussia princeps | Gaussia luciferase |
| Gaussia-Dura luciferase | |
| Cypridina noctiluca | Cypridina luciferase |
| Cypridina hilgendorfii | Cypridina (Vargula) luciferase |
| Metridia longa | Metridia luciferase |
| Oplophorus gracilorostris | OLuc |

3. Assay Controls

Methods for detecting BD typically include one or more controls. Controls can be samples of genetic material, for example, genomic DNA, obtained from a subject who does not have BD. A “test” assay using a “native” or “normal” CPG2 promoter sequence that does not carry one or more genetic variants associated with BD can be carried out in parallel or combination with one or more control assays which help improve the accuracy of determining the transcription rate/activity of the promoter of CPG2 in a sample. For example, a control is a “normal” GPG2 promoter sequence, that is used to express a reporter protein and to assess the relative expression levels of reporter genes by mutant variants of CPG2. An exemplary “normal” CPG2 promoter region does not include one or more of the SNPs associated with BD, including rs924872285, rs4530871, and rs4523096. Therefore, in some embodiments, expression of a reporter protein by the “control CPG2” promoter is carried out in parallel with the test assay in which the CPG2 promoter purified from genomic DNA of a subject suspected of having BD is assessed.

In some embodiments, a subject who is identified as having BD is a positive control for use in methods for diagnosing BD in a subject. For example, in some embodiments, a CPG2 nucleic acid sequence isolated from a sample from a subject who is identified as having BD is a positive control for use in methods for diagnosing BD in a subject. In some embodiments, a subject who is identified as not having BD is a negative control for use in methods for diagnosing BD in a subject. For example, in some embodiments, a CPG2 nucleic acid sequence isolated from a sample or database from a subject who is identified as not having BD is a negative control for use in methods for diagnosing BD in a subject.

C. Methods for Drug Screening

There is a lack of effective treatments available for bipolar disorder. In addition, the available therapeutic strategies exhibit varied degrees of efficacy in different patients, with little guidance to determine the likelihood of successful treatment prior to administering drugs. The systems and methods are useful to investigate the activity or applicability of one or more test compounds to treat or alleviate one or more symptoms of a neuropsychiatric disorder, such as a heritable mood disease or disorder, for example, bipolar disorder (BD). Therefore, in some embodiments, the methods include one or more steps for assessing transcription of CPG2 in the presence of one or more active agents, where the transcription of CPG2 in the presence of the active agent is assessed by comparison with transcription of CPG2 in the absence of the active agent. In an exemplary embodiment, an active agent is selected if it is effective to reverse or reduce the impact of one or more SNPs within the putative promoter region of the human CPG2 gene, including rs924872285, rs4530871, and rs4523096.

In an exemplary embodiment, the nucleic acid sequence of the promoter region of the candidate plasticity gene 2 (CPG2) gene is isolated from the genomic DNA of a subject and introduced into an expression vector, for example, a vector including a reporter gene. The vector is transfected, optionally together with one or more control vectors, into a target cell that is cultured in vitro. One or more test compounds are applied to the culture system and evaluated for the ability to increase the expression of the reporter gene in a test sample from a subject identified as having BD. For example, in some embodiments, an active agent is selected as being a potential therapeutic agent effective against BD when the active agent enhances the expression of the reporter protein as compared to expression of the reporter protein in the absence of the agent. Preferably, the expression of the reporter protein is determined by bioluminescence. Therefore, in preferred embodiments, an active agent is selected as being a potential therapeutic agent effective against BD when the active agent enhances the bioluminescence of the test sample as compared to that of the test sample in the absence of the agent.

Exemplary methods for identifying an active agent as a potential therapeutic agent for treatment of bipolar disorder include
(a) assessing activity of the CPG2 promoter, or a fragment of the CPG2 promoter of a subject,
wherein the subject is selected as having bipolar disorder or being at risk of bipolar disorder when the subject has reduced transcription of CPG2 as compared to a control; and
(b) assessing activity of the CPG2 promoter, or a fragment of the CPG2 promoter in the presence of one or more active agents,
wherein activity of the CPG2 promoter or the fragment thereof in the presence of the active agent is assessed by comparison with activity of the fragment of CPG2 promoter in the absence of the active agent; and
(c) selecting the active agent as a therapeutic agent for treatment of bipolar disorder when activity of the CPG2 promoter or the fragment thereof is increased (e.g., increase in transcription of CPG2) in the presence of the active agent.

In further embodiments, the methods include one or more steps for selecting the optimal amount of an active agent. For example, in some embodiments, the methods select an optimal amount of an active agent by titrating the amount of the active agent to determine the amount of the agent required to increase transcription of CPG2.

In some embodiments, the methods identify an active agent as a potential therapeutic agent for treatment of bipolar disorder when the agent exhibits an effect on the region of the CPG2 1 kilobase upstream of the transcription start site of CPG2 including the 5′ untranslated region of a subject.

In some embodiments, the methods identify an active agent as a potential therapeutic agent for treatment of bipolar disorder when the agent exhibits an effect on the region of the CPG2 promoter spanning 200 base pairs from between 800 bp and 600 bp upstream of the transcription start site of CPG2 of a subject. For example, in some embodiments the methods include the steps of
(a) assessing activity of the CPG2 promoter that includes a fragment of the CPG2 promoter spanning 200 base pairs from between 800 bp and 600 bp upstream of the transcription start site of CPG2 of a subject,
wherein the subject is selected as having bipolar disorder or being at risk of bipolar disorder when the subject has reduced transcription of CPG2 as compared to a control; and
(b) assessing activity of the CPG2 promoter that includes a fragment of the CPG2 promoter spanning 200 base pairs from between 800 bp and 600 bp upstream of the transcription start site of CPG2 in the presence of one or more active agents,
wherein activity of the fragment of the CPG2 promoter in the presence of the active agent is assessed by comparison with activity of the fragment of CPG2 promoter in the absence of the active agent; and
(c) selecting the active agent as a therapeutic agent for treatment of bipolar disorder when activity of the CPG2 promoter that includes a fragment of the CPG2 promoter spanning 200 base pairs from between 800 bp and 600 bp upstream of the transcription start site of CPG2 is increased in the presence of the active agent.

In further embodiments, the methods include one or more steps for selecting the optimal amount of an active agent that exhibits an effect on the region of the CPG2 promoter spanning 200 base pairs from between 800 bp and 600 bp upstream of the transcription start site of CPG2. For example, in some embodiments, the methods select an optimal amount of an active agent that exhibits an effect upon the region of the CPG2 promoter spanning 200 base pairs from between 800 bp and 600 bp upstream of the transcription start site of CPG2 by titrating the amount of the active agent to determine the amount of the agent required to increase transcription of CPG2. An exemplary active agent that exhibits an effect on the region of the CPG2 promoter spanning 200 base pairs from between 800 bp and 600 bp upstream of the transcription start site of CPG2 is lithium (Li). Therefore, in some embodiments, the methods include one or more steps for selecting the optimal amount of Li for use in treatment of bipolar disorder. In certain embodiments, the methods screen one or more active agents to be used in combination with an existing therapeutic agent, to identify a specific preferred combination therapy for treatment of mood disorders, such as BD. In some embodiments, the methods screen one or more active agents to be used in combination with lithium (Li). Exemplary methods for identifying an active agent as a potential combination therapeutic agent with an existing therapeutic agent for treatment of bipolar disorder include
(a) assessing activity of the CPG2 promoter that includes a fragment of the CPG2 promoter spanning 200 base pairs from between 800 bp and 600 bp upstream of the transcription start site of CPG2 of a subject,
wherein the subject is selected as having bipolar disorder or being at risk of bipolar disorder when the subject has reduced transcription of CPG2 as compared to a control; and
(b) assessing activity of the CPG2 promoter that includes a fragment of the CPG2 promoter spanning 200 base pairs from between 800 bp and 600 bp upstream of the transcription start site of CPG2 in the presence of one or more active agents and an existing or known drug (e.g., lithium),
wherein activity of the fragment of the CPG2 promoter in the presence of the active agent and the existing or known drug (e.g., lithium) is assessed by comparison with activity of the fragment of CPG2 promoter in the presence of the existing or known drug only (e.g., lithium); and
(c) selecting the active agent as a combination therapeutic agent to be used with the existing or known drug only (e.g., lithium) for treatment of bipolar disorder when activity of the CPG2 promoter that includes a fragment of CPG2 promoter is increased in the presence of the active agent.

In further embodiments, the methods include one or more steps for selecting the optimal amount of a combination therapeutic agent to be used with an existing or known drug.

1. Active Agents

The methods screen one or more active agents for increasing (i.e., reversing the reduction of) the transcription and expression of reporter proteins associated with BD in the described in vitro transcription assays. In an exemplary embodiment, the active agent is selected if it is effective to reverse or reduce the impact of one or more SNPs within the putative promoter region of the human CPG2 gene, including rs924872285, rs4530871, and rs4523096.

The active agents can be proteins, carbohydrates, polypeptides, amino acids, nucleic acids, lipids, small molecules, microparticles or nanoparticles. In some embodiments, the active agents are drugs, such as drugs known to be associated with treatment of neuropsychiatric diseases or disorders. For example, in some embodiments active agents are therapeutic agents that are known to be effective in treating one or more symptoms of BD. Exemplary drugs include mood stabilizers such as lithium (ESKALITH®; LITHOBID®), valproic acid (DEPAKENE®), divalproex sodium (DEPAKOTE®), carbamazepine (TEGRETOL®, EQUETRO®, others) and lamotrigine (LAMICTAL®); Antipsychotics such as olanzapine (ZYPREXA®), risperidone (RISPERDAL®), quetiapine (SEROQUEL®), aripiprazole (ABILIFY®) ziprasidone (GEODON®), lurasidone (LATUDA®) or asenapine (SAPHRIS®); Antidepressants, such as Symbyax, fluoxetine and olanzapine; and Anti-anxiety medications, such as Benzodiazepines. A preferred active agent is Lithium.

a. Lithium (Li)

In some embodiments, the one or more agent that is used for upregulation of CPG2 activity is lithium. Lithium treatment is the “gold standard” of treatment for preventing recurrences in bipolar disorder, both types I (with mania and major depression) and II (with depression and hypomania) and is used mostly as a long-term treatment to prevent mood-disorder recurrences. Lithium is in a class of medications called antimanic agents. It works by decreasing abnormal activity in the brain. It has also shown activity in enhancing the expression and activity of the CPG2 protein.

Lithium is commercially available under the tradename ESKALITH®; LITHOBID®, and is available in tablets for oral administration of 100 mg to 450 mg, in capsules and as a solution.

In some embodiments, lithium is used to confirm a diagnosis of bipolar disorder and/or predict treatment efficacy, for example, by enhancing the expression and activity of the CPG2 promoter from a subject with BD. In other embodiments, lithium, or a salt thereof is used as a standard or control in a simultaneous assay to identify one or more additional active agents that can also enhance the expression and activity of the CPG2 protein from a subject with BD.

2. Selection of Subjects for Treatment

In some embodiments, the methods to select a subject as being likely to benefit from therapeutic treatment with one or more active agents for preventing or ameliorating the reduction in the transcription and expression of reporter proteins associated with BD. For example, in some embodiments, the methods employ the described in vitro transcription assays to determine the efficacy of an active agent for reversing or ameliorating BD in the subject from whom the genomic DNA sample was obtained.

Therefore, in some embodiments, the methods identify a subject who will benefit from treatment with an active agent that is identified as reversing or ameliorating the reduced transcription and expression of reporter proteins associated with BD, as identified in the described in vitro transcription assays.

Exemplary methods for identifying a subject with bipolar disorder as being likely to receive therapeutic benefit from treatment with one or more active agents include
(a) assessing the activity of the promoter of the candidate plasticity gene 2 (CPG2) from the subject,
wherein the subject is selected as having bipolar disorder or being at risk of bipolar disorder when the subject has reduced activity of the CPG2 promoter as compared to a control;
(b) assessing the activity of the CPG2 promoter in the presence of one or more active agents,
wherein the activity of the CPG2 promoter in the presence of the active agent is assessed by comparison with the activity of the CPG2 promoter in the absence of the active agent; and
(c) selecting the subject with bipolar disorder as being likely to receive therapeutic benefit from treatment with the active agents when the activity of the CPG2 promoter is increased in the presence of the active agent.

In preferred embodiments, the CPG2 promoter region includes 1 kilobase upstream of the transcription start site of CPG2 including the 5′ untranslated region, more preferably a fragment of the CPG2 promoter spanning 200 base pairs from between 800 bp and 600 bp upstream of the transcription start site of CPG2 of the subject.

In other embodiments, the methods include one or more steps for (i) selecting an active agent as being a potential therapeutic agent effective to treat bipolar disorder for the selected subject. For example, in some embodiments the methods include
(a) assessing the activity of the promoter of candidate plasticity gene 2
(CPG2) from a subject both in the presence and in the absence of a therapeutic agent; and
(b) selecting the subject for treatment for bipolar disorder with the therapeutic agent when the activity of the CPG2 promoter is increased in the presence of the therapeutic agent.

In further embodiments, the methods include one or more steps for selecting the optimal amount of an active agent for treatment of a subject with bipolar disorder. For example, in some embodiments, the methods select an optimal amount of an active agent by titrating the amount of the active agent to determine the amount of the agent required to increase the activity of the CPG2 promoter from the subject. In a preferred embodiment, the therapeutic agent is lithium (Li), or a salt thereof. All the methods can include one or more additional steps of treating a subject.

D. Methods for Treating Bipolar Disorders

Methods of treatment of bipolar disorders are also provided.

Any of the active agent drug candidates identified using the described screening methods can be used in a method of treating the symptom(s) or condition(s) for which the drug showed effectiveness. Therefore, in some embodiments, drug candidates identified using the described screening methods can be used in a method of treating bipolar disorder in a subject in need thereof. An exemplary method for diagnosing and treating a subject for bipolar disorder includes one or more steps of
(a) assessing the activity of the candidate plasticity gene 2 (CPG2) promoter, or a fragment thereof from a subject,
wherein the subject is selected as having bipolar disorder or being at risk of bipolar disorder when the subject has reduced activity of the CPG2 promoter, or the fragment thereof as compared to a control;
(b) assessing the activity of the CPG2 promoter or the fragment thereof from the subject in the presence of one or more active agents,
wherein the activity of the CPG2 promoter or the fragment thereof from the subject in the presence of the active agent is assessed by comparison with the activity of the CPG2 promoter or the fragment thereof in the absence of the active agent;
(c) selecting the active agent as a therapeutic agent for treatment of bipolar disorder when the activity of the CPG2 promoter or the fragment thereof is increased in the presence of the active agent; and
(d) treating the subject for bipolar disorder,
wherein the treating includes administering to the subject an effective amount of the active agent(s) to treat or prevent one or more symptoms of bipolar disorder in the subject.

1. Drug Screening-Guided Therapy

The methods can be used for drug screening and/or drug validation, and to identify a specific preferred combination therapy for treatment of mood disorders, such as BD. Therefore, in some embodiments, methods of treating a subject for BD include identifying the subject as having, or as being at risk of having BD, selecting one or more drugs identified as has therapeutic efficacy in an in vitro transcription assay, and administering the one or more drugs to the subject in an amount effective to treat or prevent BD in the subject.

Variables that must be considered include dosage, timing of administration, and selection of drugs. Therapies including administering one or more active agents identified according to the methods for selecting effective therapeutic agents are provided. In a preferred embodiment, the methods administer lithium, optionally in combination with one or more additional active agents, for the treatment or prevention of BD.

The treatment can include administering to a subject in need thereof an effective amount of lithium, optionally in combination with one or more additional therapeutic, prophylactic, or diagnostic agents.

Therapy is typically involved in upregulation of CPG2 activity, for example, increasing the expression of the CPG2 protein. The upregulation of CPG2 activity may help reduce or prevent one or more symptoms of the mood disorder, such as reducing one or more symptoms of BD. Dosage and timing of administration is important. For example, in some embodiments, a subject has been diagnosed with BD and the methods select a suitable therapeutic agent, or a suitable dosage or therapeutic regimen. Therefore, in some embodiments, the methods include one or more steps of
(a) assessing the activity of the candidate plasticity gene 2 (CPG2) promoter or a fragment thereof from a subject both in the presence and in the absence of an active agent candidate drug in vitro;
(b) selecting the subject for treatment for bipolar disorder with the therapeutic agent when the activity of the CPG2 promoter or a fragment thereof from the subject is increased in the presence of the active agent candidate drug in vitro; and, optionally
(c) selecting the optimal amount of the active agent candidate drug, where selecting the amount includes titrating the amount of the active agent to identify an amount effective to increase the activity of the CPG2 promoter or the fragment thereof from the subject in vitro equivalent to that of a normal control CPG2
(d) performing a calculation to extrapolate the dosage required to treat the subject in vivo based on the amount determined in vitro; and
(e) treating the subject by administering to the subject an amount of the active agent candidate drug identified in (d).

2. Methods of Administration and Dosage Regimes

The methods of treatment include administering to a subject in need thereof, an effective amount of an active agent identified as being effective in the treatment of BD, to treat or prevent BD or symptom(s) thereof, or to produce a desired physiological change in the subject. A desired physiological change is an enhanced transcription of CPG2 in the subject, for example, by ameliorating or reducing the effects of one or more SNPs in the promoter region of CPG2 in the subject. In an exemplary embodiment, the amount of active agent is effective to reverse or reduce the impact of one or more SNPs within the putative promoter region of the human CPG2 gene, including rs924872285, rs4530871, and rs4523096.

In preferred embodiments, the methods treat or prevent BD in a subject determined as having BD by the described methods by administering to the subject an amount of lithium to treat of prevent the BD. When administered to treat a mood disorder such as BD, the amount of lithium is the amount effective to reverse or reduce the impact of one or more SNPs within the putative promoter region of the human CPG2 gene, including rs924872285, rs4530871, and rs4523096 in vivo, by extrapolating the amount of an active agent required to increase transcriptional efficacy of the CPG2 promoter in vitro. In other embodiments, the amount of lithium can be the amount effective to reduce one or more symptoms of BD in the subject, for example, psychiatric symptoms selected from hallucinations, suicidality, cognitive deficits, mania, or hypomania (flight of ideas, elevated mood, and pressured speech), depressed mood, concentration problems, anxious distress, melancholy, and psychosis.

The efficacy of administration of a particular dose of a therapeutic drug or agent according to the methods described can be determined by evaluating the particular forms of the medical history, signs, symptoms, and objective laboratory tests that are known to be useful in evaluating the status of a subject in need treatment of a given diseases and/or conditions. These signs, symptoms, and objective laboratory tests will vary, depending upon the disease or condition being treated or prevented, as will be known to any clinician who treats such patients or a researcher conducting experimentation in this field. For example, if, based on a comparison with an appropriate control group and/or knowledge of the usual progression of the disease in the general population or the individual: (1) a subject's physical condition is shown to be improved, (2) the progression of the disease or condition is shown to be stabilized, or slowed, or reversed, or (3) the need for other medications for treating the disease or condition is lessened or obviated, then a particular treatment regimen will be considered efficacious. An effective amount of therapeutic agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

a. Combination Treatments

In some embodiments, a combination of two or more active agents are administered to the same subject in an amount effective to treat or prevent one or more symptoms of BD in the subject. For example, in some embodiments, the combination of a first and second active agent has the same or greater therapeutic efficacy in treating BD in a subject than the result achieved by administering either the first or second agent alone. A combination may be partially or completely additive of the results achieved by the individual components alone. In some embodiments, the result achieved by the combination is more than additive of the results achieved by the individual components alone. In some embodiments, the effective amount of one or both agents used in combination is lower than the effective amount of each agent when administered separately. In some embodiments, the amount of one or both agents when used in the combination therapy is sub-therapeutic when used alone.

A treatment regimen of the combination therapy can include one or multiple administrations of one or more of the compounds. For example, in some embodiments, a treatment regimen of the combination therapy includes one or multiple administrations of lithium and one or multiple administrations of another active agent. In certain embodiments, lithium and an additional active agent are administered to a subject simultaneously. Where lithium and an additional active agent are administered at the same time, they can be in the same pharmaceutical composition or separate pharmaceutical compositions.

In some embodiments, lithium and an additional active agent are administered sequentially, for example, in two or more different pharmaceutical compositions. In certain embodiments, the lithium is administered prior to the first administration of the additional active agent. In other embodiments, the additional active agent is administered prior to the first administration of lithium. For example, in some embodiments the lithium and an additional active agent are administered to a subject on the same day. Alternatively, the lithium and additional active agent are administered to the subject on different days.

The lithium can be administered at least 1, 2, 3, 5, 10, 15, 20, 24 or 30 hours or days prior to or after administering of the additional active agent. Alternatively, the additional active agent can be administered at least 1, 2, 3, 5, 10, 15, 20, 24 or 30 hours or days prior to or after administering of the lithium. In certain embodiments, additive or more than additive effects of the administration of lithium in combination with the additional active agent is evident after one day, two days, three days, four days, five days, six days, one week, or more than one week following administration. In a preferred embodiment, the interval between treatment is 24 h and maximum treatment duration is day 4 to day 14 (10 days).

Dosage regimens or cycles of the agents can be completely or partially overlapping or can be sequential. For example, in some embodiments, all such administration(s) of lithium occur before or after administration of the additional active agent. Alternatively, administration of one or more doses of lithium can be temporally staggered with the administration of the additional active agent to form a uniform or non-uniform course of treatment whereby one or more doses of lithium are administered, followed by one or more doses of the additional active agent, followed by one or more doses of lithium, followed by one or more doses of the additional active agent, etc., all according to whatever schedule is selected or desired by the researcher or clinician administering the therapy.

b. Therapeutic Controls

The therapeutic result of the one or more active agents for treatment or prevention of bipolar disorder can be compared to a control. Suitable controls are known in the art and include, for example, untreated cells or an untreated subject with bipolar disorder. A typical control is a comparison of the bipolar disorder, or symptom of the bipolar disorder in a subject prior to and after administration of the active agent. The condition or symptom can be a biochemical, molecular, physiological, or pathological readout. For example, the effect of the active agent on a particular symptom, pharmacologic, or physiologic indicator can be compared to an untreated subject, or the condition of the subject prior to treatment. In some embodiments, the symptom, pharmacologic, or physiologic indicator of bipolar disorder is measured in a subject prior to treatment, and again one or more times after treatment is initiated. In some embodiments, the control is a reference level, or average determined based on measuring the symptom, pharmacologic, or physiologic indicator in one or more subjects that do not have bipolar disorder (e.g., healthy subjects). In some embodiments, the effect of the treatment is compared to a conventional treatment for bipolar disorder that is known the art.

3. Formulations

In some embodiments, the methods include administering to a subject with BD one or more therapeutic agents formulated as a pharmaceutical composition. Pharmaceutical compositions can be for administration by parenteral (intramuscular, intraperitoneal, intravenous (IV) or subcutaneous injection), enteral, or transmucosal (nasal, pulmonary, vaginal, rectal, or sublingual) routes of administration or using bioerodible inserts and can be formulated in dosage forms appropriate for each route of administration.

Drugs can be formulated for immediate release, extended release, or modified release. A delayed release dosage form is one that releases a drug (or drugs) at a time other than promptly after administration. An extended-release dosage form is one that allows at least a twofold reduction in dosing frequency as compared to that drug presented as a conventional dosage form (e.g., as a solution or prompt drug-releasing, conventional solid dosage form). A modified release dosage form is one for which the drug release characteristics of time course and/or location are chosen to accomplish therapeutic, or convenience objectives not offered by conventional dosage forms such as solutions, ointments, or promptly dissolving dosage forms. Delayed release and extended-release dosage forms and their combinations are types of modified release dosage forms.

Transdermal formulations may also be prepared. These will typically be gels, ointments, lotions, sprays, patches, or microneedle devices. Transdermal formulations can include penetration enhancers.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

III. Kits

Kits for use with the described methods are also provided. The kits for the diagnosing bipolar disorder in a subject based on the activity of the CPG2 promoter from the subject typically include one or more reagents for performing an in vitro transcription assay, optionally reagents for cloning the CPG2 promoter region from genomic DNA into an expression vector configured to express a reporter protein controlled by the cloned CPG2 promoter, and one or more control expression vectors. Kits for the PCR-based methods typically include reagents for PCR. In some embodiments, the kits include one or more reagents for transfecting cells, and/or one or more reagents for extracting or purifying genomic DNA from cells or from a biological sample. In some embodiments, the kits include apparatus for obtaining a sample from a subject, such as a syringe, a pipette, a needle, a swab, a flask, or a vial.

Reagents can be, for example, buffers, primers, enzymes, dNTPs, carrier RNA, and other active agents and organics that facilitate various steps of the disclosed reactions. The kits can also include instructions for use. In some embodiments, the kits also include one or more active agents for use in assessing the effect of the active agent on the activity of a cloned CPG2 promoter. An exemplary active agent is lithium.

The present invention will be further understood by reference to the following non-limiting examples.

### EXAMPLES

### Example 1: Non-Reference SNP Alleles in the CPG2 Promoter Region and their Effect on Gene Expression

Methods

Molecular Cloning

Putative promoter DNA regions from purified control or patient gDNA samples were amplified using LongAmp Taq polymerase and proofreading Q5 polymerase (NEB). PCR products from promoter regions were separated on agarose gels, excised, purified and cloned into the pGL3-Basic vector (Promega) in front of the Firefly Luciferase (Luc+) gene using KpnI and XhoI restriction sites.

Neuronal Cultures

Rat cortical or hippocampal cultures were prepared using known methods.

Luciferase Assay

At 8 DIV, cortical neurons were transfected by calcium phosphate precipitation for 1 h with 1 μg of various Firefly Luciferase plasmids and 1.2 μg of the normalization control pRL-TK Renilla Luciferase (RLuc) vector plasmid (Pro-mega). At 14 DIV, neurons were lysed and assessed using a Dual-Luciferase® Reporter Assay System (Pro-mega) according to the manufacturer's protocol. Plates were read on an EnSpire® Multimode Plate Reader (PerkinElmer). Where indicated, neurons were treated with lithium chloride (2 mM) for 24 h before cells were lysed and assayed. Relative Luciferase activity was statistically compared using Student's two-tailed t test and one-way ANOVAs with Tukey's post-hoc tests for multiple comparisons, as appropriate.

Results

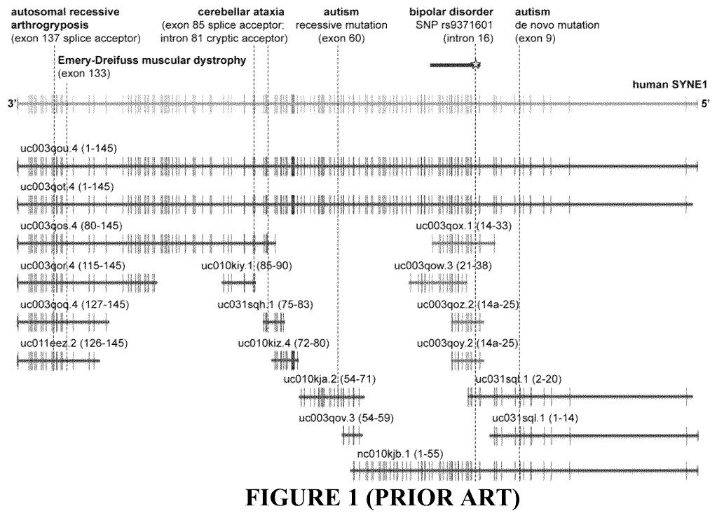

An earlier study identified CPG2 gene regions with promoter activity using a Luciferase gene expression assay in cultured cortical neurons and described the effect of SNP alleles rs4523096[T] and rs4530871[T] combined, on relative luciferase activity of the 5′UTR i16 region (Rathje M et al., Mol Psychiatry 26, 508-523 (2021)), published in 2019 (FIG. 1).

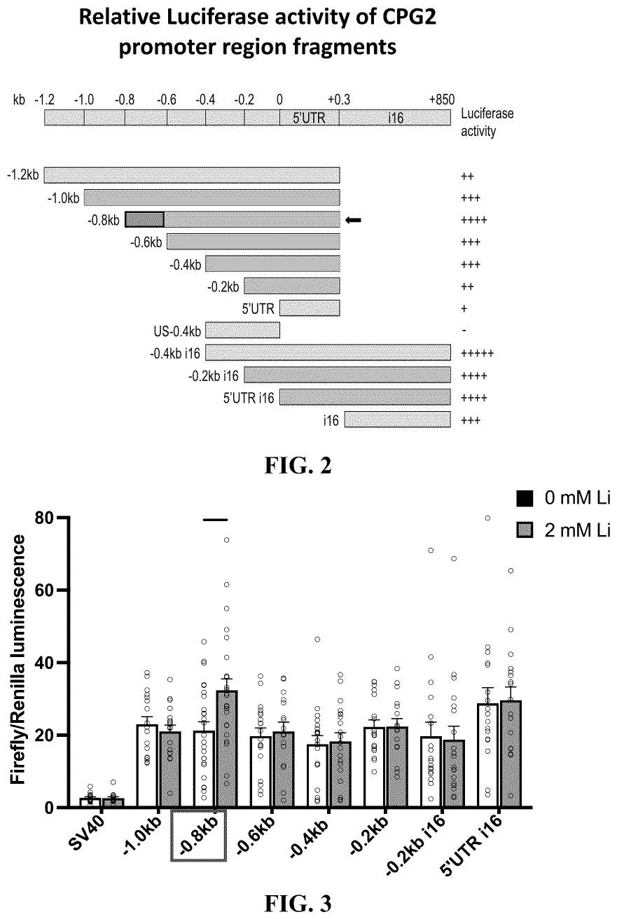

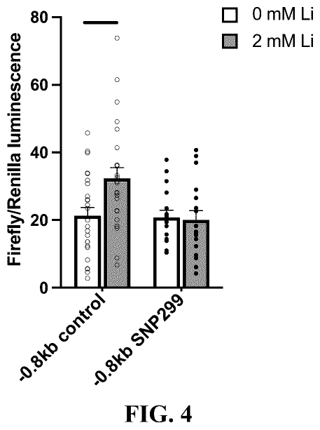

Among several CPG2 promoter regions that were examined in the dual-Luciferase reporter assay for their promoter activity, the −1.0 kb, −0.8 kb, −0.6 kb, −0.4 kb, −0.2 kb, −0.2 kb i16, and 5′UTR i16 fragments showed some of the most robust promoter activity, as shown in FIG. 2. In the current study, these promoter fragments were tested for the impact of lithium treatment on CPG2 transcription. The data show that the −0.8 kb CPG2 promoter region, but not other regions, is responsive to lithium treatment (FIG. 3, **p<0.01, 2-way ANOVA with the Sidak's multiple comparisons test). There is no difference in Luciferase activity across groups in neurons transfected with the SV40 control promoter demonstrating that lithium treatment does not impact the general transcriptional activity. The observation that the −0.8 kb, but not −0.6 kb, promoter region is responsive to lithium treatment suggests that the 200 bp region that belongs to the −0.8 kb but not the −0.6 kb region contains essential gene sequences that may be important for the response to the BD treatment lithium. Moreover, the presence of a patient SNP within this 200 bp region (rs118135299) blunted the LiCl influence on CPG2 transcription, as shown in FIG. 4.

Modifications and variations of the method of the present invention will be obvious to those skilled in the art from the foregoing description and are intended to come within the scope of the appended claims.

We claim:

1. A method for diagnosing bipolar disorder in a subject, comprising
(a) measuring the activity of the promoter of the candidate plasticity gene 2 (CPG2), or a fragment of the promoter of CPG2 from the subject, comprising a nucleic acid sequence between about 800 bp and about 600 bp upstream of the transcription start site of CPG2.
(b) wherein the subject is selected as having bipolar disorder or as being at risk of bipolar disorder when the activity of the CPG2 promoter, or fragment thereof from the subject is less than that of a control CPG2 promoter, or fragment thereof.

2. The method of claim 1, further comprising
(c) assessing the effect of one or more active agents upon the activity of the CPG2 promoter or fragment thereof from the subject,
wherein the effect of the active agent is determined by comparison with the activity of the CPG2 promoter or fragment thereof in the absence of the active agent.

3. The method of claim 2, wherein assessing the activity of the CPG2 promoter or fragment thereof comprises an in vitro transcription assay.

4. The method of claim 3, wherein the in vitro transcription assay comprises one or more steps of
(i) obtaining genomic DNA including the promoter of CPG2 from the subject or obtaining DNA sequence information from the CPG2 region from a patient database that has genomic data of the subject;
(ii) introducing the nucleic acid sequence of the CPG2 promoter or fragment thereof from the subject into an expression vector configured to express a reporter protein controlled by the CPG2 promoter;
(iii) expressing the reporter protein controlled by the CPG2 promoter in one or more cell lines; and
(iv) quantitating the expressed reporter protein to assess the activity of the CPG2 promoter or fragment thereof from the subject by reference to a control.

5. The method of claim 1, wherein the fragment of the CPG2 promoter comprises a nucleic acid of about 200 base pairs in size.

7. The method of claim 1, wherein the fragment of the CPG2 promoter comprises a nucleic acid sequence from between greater than 1 base pair and 800 base pairs, inclusive, upstream of the transcription start site of CPG2.

8. The method of claim 1, wherein the fragment of the CPG2 promoter comprises a nucleic acid sequence of between greater than about 1 base pair and about 1,000 base pairs, inclusive, upstream of the transcription start site of CPG2.

9. The method of claim 1, wherein the nucleic acid sequence of the CPG2 promoter, or fragment thereof, from the subject comprises one or more single nucleotide polymorphisms (SNPs).

10. The method of claim 9, wherein the one or more single nucleotide polymorphisms (SNPs) are selected from the group consisting of rs118135299, rs924872285, rs4530871, and rs4523096.

11. The method of claim 9, further comprising one or more steps to identify an SNP within the CPG2 promoter, or fragment thereof as being associated with bipolar disorder comprising identifying SNPs within the CPG2 promoter, or fragment thereof from the subject, and
identifying an SNP as being associated with bipolar disorder when the activity of the CPG2 promoter, or fragment thereof from the subject is less than that of a control CPG2 promoter, or fragment thereof.

12. The method of claim 11, wherein the control comprises one or more expression products regulated by a CPG2 promoter without SNPs.

13. The method of claim 11, wherein the control comprises a CPG2 promoter comprising the nucleic acid sequence of SEQ ID NO:1.

14. The method of claim 4, wherein the reporter protein is expressed in cultured cortical neuron cells.

15. The method of claim 4, wherein the expressed reporter protein is quantitated using a luciferase reporter assay.

16. The method of claim 15, wherein the luciferase reporter assay is selected from the group consisting of a Single Luciferase Reporter Assay, a Dual Non-Secreted Luciferase Reporter Assay, a Dual Secreted Luciferase Reporter Assay, a Real Time Luciferase Reporter Assay, and a Multicolor Luciferase Reporter Assay.

17. The method of claim 16, wherein the assay is a Dual Non-Secreted Luciferase Reporter Assay, and wherein the CPG2 promoter controls expression of firefly luciferase and the control promoter controls expression of Renilla luciferase within the same cells.

18. The method of claim 2, further comprising
(c) selecting the one or more active agents as a potential therapeutic agent when the activity of the CPG2 promoter, or fragment thereof from the subject is increased in the presence of the active agent.

19. The method of claim 18, wherein the active agent increases the activity of the CPG2 promoter, or fragment thereof from the subject to be equivalent to that of the control.

20. The method of claim 18, wherein the active agent is lithium, or a salt thereof.

21. The method of claim 18, further comprising
(d) treating the subject by administering to the subject an effective amount of the active agent to treat or prevent one or more symptoms of bipolar disorder in the subject.

22. A method for selecting a subject having bipolar disorder for treatment with a therapeutic agent, comprising
(a) assessing the activity of the promoter of candidate plasticity gene 2 (CPG2), or a fragment thereof from the subject in vitro both in the presence and in the absence of the therapeutic agent; and
(b) selecting the subject for treatment for bipolar disorder with the therapeutic agent when the activity of the CPG2 promoter or fragment thereof from the subject is increased in the presence of the therapeutic agent.

23. The method of claim 22, wherein the fragment of the CPG2 promoter comprises a fragment of the CPG2 promoter comprising a nucleic acid of about 200 base pairs in size.

24. The method of claim 22, wherein the fragment of the CPG2 promoter comprises a nucleic acid sequence spanning about 200 base pairs between about 800 bp and about 600 bp upstream of the transcription start site of CPG2.

25. The method of claim 22, wherein the fragment of the CPG2 promoter comprises a nucleic acid sequence from between greater than 1 base pair and 1,000 base pairs, inclusive, upstream of the transcription start site of CPG2.

26. The method of claim 22, wherein the therapeutic agent is lithium, or a salt thereof.

27. The method of claim 22, further comprising
(c) selecting an optimal dose of the therapeutic agent,
wherein selecting the optimal dose comprises titrating the amount of the active agent to determine the amount of the agent required to increase the activity of the CPG2 promoter equivalent to that of a normal control CPG2 promoter in the absence the active agent in vitro, and extrapolating the amount required to treat the subject in vivo.

28. The method of claim 1, wherein the subject is an individual who has symptoms characteristic of Bipolar I disorder, Bipolar II disorder, or Cyclothymic disorder.

29. The method of claim 1, wherein the subject has one or more psychiatric symptoms selected from the group consisting of hallucinations, suicidality, cognitive deficits, mania, or hypomania (flight of ideas, elevated mood, and pressured speech), depressed mood, concentration problems, anxious distress, melancholy, and psychosis.

30. The method of claim 1, wherein the subject does not have any psychiatric symptoms of BD.

31. The method of claim 1, wherein the subject does not have one or more of Schizophrenia, Alzheimer's Disease, Panic Disorder and Major Depression.

32. The method of claim 1, wherein the subject has a family history of BD.

33. A kit for identifying bipolar disorder in a subject, comprising
(a) reagents for assessing the activity of the promoter of candidate plasticity gene 2 (CPG2) or a fragment thereof in the subject;
optionally, (b) a control sample, comprising one or more expression vectors regulated by the promoter of CPG2 without single nucleotide polymorphisms (SNPs) associated with bipolar disorder or normal ranges for the activity of the promoter of candidate plasticity gene 2 (CPG2) or a fragment thereof in the subject; and
(c) instructions for the method of claim 1.

34. The kit of claim 33, further comprising
(d) one or more active agents; and
(e) instructions for the method of claim 2.

35. The kit of claim 33, wherein the one active agent is lithium, or a salt thereof.

36. The kit of claim 33, wherein the fragment of the CPG2 promoter comprises the fragment of the CPG2 promoter comprises a nucleic acid of about 200 base pairs in size.

37. The kit of claim 33, wherein the fragment of the CPG2 promoter comprises a nucleic acid sequence spanning about 200 base pairs between about 800 bp and about 600 bp upstream of the transcription start site of CPG2.

38. The kit of claim 33, wherein the fragment of the CPG2 promoter comprises a nucleic acid sequence from between 1 base pair and 1,000 base pairs, inclusive, upstream of the transcription start site of CPG2.
